(19) United States
(12) Patent Application Publication
(10) Pub. No.: US 2005/0261598 A1
(43) Pub. Date: Nov. 24, 2005

(54) PATCH SENSOR SYSTEM FOR MEASURING VITAL SIGNS

(75) Inventors: Matthew John Banet, Del Mar, CA (US); Zhou Zhou, La Jolla, CA (US)

Correspondence Address: MATTHEW J. BANET, 6540 LUSK BLVD., C200, SAN DIEGO, CA, 92121, US

(73) Assignee: TRIAGE WIRELESS, INC., San Diego, CA (US)

(21) Appl. No.: 11/160,957

(22) Filed: Jul. 18, 2005

Related U.S. Application Data
(63) Continuation-in-part of application No. 10/906,315, filed on Feb. 14, 2005.
Application No. 10/906,315 is a continuation-in-part of application No. 10/709,014, filed on Apr. 7, 2004.

(51) Int. Cl. A61B 5/02

(52) U.S. Cl. 600/513; 600/483

(57) ABSTRACT

The invention provides a system for measuring vital signs from a patient that includes: 1) a first adhesive patch featuring a first electrode that measures a first electrical signal from the patient; 2) a second adhesive patch featuring a second electrode that measures a second electrical signal from the patient; 3) a third adhesive patch, in electrical communication with the first and second adhesive patches, featuring an optical system that measures an optical waveform from the patient; and 4) a controller that receives and processes the first and second electrical signals and the optical waveform to determine the patient's vital signs.

### CROSS REFERENCES TO RELATED APPLICATION

This application is a continuation-in-part of U.S. patent application Ser. No. 10/906,315, filed Feb. 14, 2005, which is a continuation-in-part application of U.S. patent application Ser. No. 10/709,014, filed on Apr. 7, 2004.

### BACKGROUND OF THE INVENTION

The present invention relates to a device, method and system for measuring vital signs, particularly blood pressure.

### DESCRIPTION OF RELATED ART

Pulse oximeters are medical devices featuring an optical module, typically worn on a patient's finger or ear lobe, and a processing module that analyzes data generated by the optical module. The optical module typically includes first and second light sources (e.g., light-emitting diodes, or LEDs) that transmit optical radiation at, respectively, red (λ˜630-670 nm) and infrared (λ˜800-1200 nm) wavelengths. The optical module also features a photodetector that detects radiation transmitted or reflected by an underlying artery.

Typically the red and infrared LEDs sequentially emit radiation that is partially absorbed by blood flowing in the artery. The photodetector is synchronized with the LEDs to detect transmitted or reflected radiation. In response, the photodetector generates a separate radiation-induced signal for each wavelength. The signal, called a plethysmograph, varies in a time-dependent manner as each heartbeat varies the volume of arterial blood and hence the amount of transmitted or reflected radiation. A microprocessor in the pulse oximeter processes the relative absorption of red and infrared radiation to determine the degree of oxygen saturation in the patient's blood. A number between 94%-100% is considered normal, while a value below 85% typically indicates the patient requires hospitalization. In addition, the microprocessor analyzes time-dependent features in the plethysmograph to determine the patient's heart rate.

Pulse oximeters work best when they attach to an appendage (e.g., a finger) that is at rest. If the finger is moving, for example, the light source and photodetector within the optical module typically move relative to the underlying artery. This generates ‘noise’ in the plethysmograph, which in turn can lead to motion-related artifacts in data describing pulse oximetry and heart rate. Ultimately this reduces the accuracy of the measurement.

Another medical device, called a sphygmomanometer, measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds the systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting strong, periodic acoustic ‘beats’ or ‘taps’ indicating that the blood is flowing past the cuff (i.e., the systolic pressure barely exceeds the cuff pressure). The minimum pressure in the cuff that restricts blood flow, as detected by the stethoscope, is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow (i.e., the diastolic pressure barely exceeds the cuff pressure).

Low-cost, automated devices measure blood pressure using an inflatable cuff and an automated acoustic or pressure sensor that measures blood flow. These devices typically feature cuffs fitted to measure blood pressure in a patient's wrist, arm or finger. During a measurement, the cuff automatically inflates and then incrementally deflates while the automated sensor monitors blood flow. A microcontroller in the automated device then calculates blood pressure. Cuff-based blood-pressure measurements such as these typically only determine the systolic and diastolic blood pressures; they do not measure dynamic, time-dependent blood pressure.

Data indicating blood pressure are most accurately measured during a patient's appointment with a medical professional, such as a doctor or a nurse. Once measured, the medical professional can manually record these data in either a written or electronic file. Appointments typically take place a few times each year. Unfortunately, in some cases, patients experience ‘white coat syndrome’ where anxiety during the appointment affects the blood pressure that is measured. For example, white coat syndrome can elevate a patient's heart rate and blood pressure; this, in turn, can lead to an inaccurate diagnoses.

Various methods have been disclosed for using pulse oximeters to obtain arterial blood pressure. One such method is disclosed in U.S. Pat. No. 5,140,990 to Jones et al., for a ‘Method Of Measuring Blood Pressure With a Photoplethysmograph’. The '990 patent discloses using a pulse oximeter with a calibrated auxiliary blood pressure measurement to generate a constant that is specific to a patient's blood pressure.

Another method for using a pulse oximeter to measure blood pressure is disclosed in U.S. Pat. No. 6,616,613 to Goodman for a ‘Physiological Signal Monitoring System’. The '613 patent discloses processing a pulse oximetry signal in combination with information from a calibrating device to determine a patient's blood pressure.

Chen et al, U.S. Pat. No. 6,599,251, discloses a system and method for monitoring blood pressure by detecting pulse signals at two different locations on a subject's body, preferably on the subject's finger and earlobe. The pulse signals are preferably detected using pulse oximetry devices, and then processed to determine blood pressure.

Schulze et al., U.S. Pat. No. 6,556,852, discloses an earpiece having an embedded pulse oximetry device and thermopile to monitor and measure physiological variables of a user.

Jobsis et al., U.S. Pat. No. 4,380,240, discloses an optical probe featuring a light source and a light detector incorporated into channels within a deformable mounting structure which is adhered to a strap. The light source and the light detector are secured to the patient's body by adhesive tapes and pressure induced by closing the strap around a portion of the body.

Tan et al., U.S. Pat. No. 4,825,879, discloses an optical probe with a T-shaped wrap having a vertical stem and a horizontal cross bar, which is utilized to secure a light source and an optical sensor in optical contact with a finger. A metallic material is utilized to reflect heat back to the patient's body and to provide opacity to interfering ambient light. The sensor is secured to the patient's body using an adhesive or hook-and-loop material.

Modgil et al., U.S. Pat. No. 6,681,454, discloses a strap composed of an elastic material that wraps around the outside of a pulse oximeter probe and is secured to the oximeter probe by attachment mechanisms such as Velcro.

Diab et al., U.S. Pat. Nos. 6,813,511 and 6,678,543, discloses a disposable optical probe that reduces noise during a measurement. The probe is adhesively secured to a patient's finger, toe, forehead, earlobe or lip, and can include reusable and disposable portions.

### BRIEF SUMMARY OF THE INVENTION

In one aspect, the invention provides a system for measuring vital signs from a patient that includes: 1) a first adhesive patch featuring a first electrode that measures a first electrical signal; 2) a second adhesive patch featuring a second electrode that measures a second electrical signal; 3) a third adhesive patch, in electrical communication with the first and second adhesive patches, featuring an optical system that measures an optical waveform; and 4) a controller that receives and processes the first and second electrical signals and the optical waveform to determine the patient's vital signs (e.g., blood pressure, heart rate, pulse oximetry, ECG, and associated waveforms).

In embodiments, the optical system features a light-emitting diode and an optical detector disposed on a same side of a substrate (e.g., a circuit board) to operate in a ‘reflection mode’ geometry. Alternatively, these components can be disposed to operate in a ‘transmission mode’ geometry.

The controller typically includes an algorithm (e.g., compiled computer code) configured to process the first and second electrical signals to generate an electrical waveform. The algorithm then processes the electrical waveform with the optical waveform to calculate a blood pressure value. For example, the controller can determine blood pressure by processing: 1) a first time-dependent feature of the optical waveform; 2) a second time-dependent feature of the electrical waveform; and 3) a calibration parameter determined by another means (e.g., a conventional blood pressure cuff or tonometer).

In embodiments, the third adhesive patch further includes a connector configured to connect to a detachable cable that, in turn, connects to the first electrode attached by the first adhesive patch and the second electrode attached by the second adhesive patch. The system can also include an additional cable that connects the third adhesive patch to the controller. Alternatively, the third adhesive patch can include a first wireless component, and the controller further includes a second wireless component configured to communicate with first wireless component. In yet another embodiment the controller is connected directly to the third adhesive patch.

The optical system typically includes a first light-emitting diode that emits radiation (e.g. red radiation) that generates a first optical waveform, and a second light-emitting diode that emits radiation (e.g., infrared radiation) that generates a second optical waveform. In this case the controller additionally includes an algorithm that processes the first and second optical waveforms to generate pulse oximetry and heart rate values. In other embodiments the controller features an algorithm that processes the first and second electrical signals to generate an ECG waveform.

In other embodiments the third adhesive patch includes a third electrode that measures a third electrical signal from the patient. In this case, the controller includes an algorithm that processes the first, second, and third electrical signals to generate an ECG waveform along with the other vital signs described above.

The invention has many advantages. In particular, it provides a single, low-profile, disposable system that measures a variety of vital signs from the patient. The system continuously measures blood pressure without using a cuff. This and other information can be easily transferred to a central monitor through a wired or wireless connection to better characterize a patient. For example, with the system a medical professional can continuously monitor a patient's blood pressure and other vital signs during their day-to-day activities. Monitoring patients in this manner minimizes erroneous measurements due to ‘white coat syndrome’ and increases the accuracy of a blood-pressure measurement. In particular, as described below, one aspect of the invention provides a system that continuously monitors a patient's blood pressure using a cuffless blood pressure monitor and an off-the-shelf mobile communication device. Information describing the blood pressure can be viewed using an Internet-based website, using a personal computer, or simply by viewing a display on the mobile device. Blood-pressure information measured continuously throughout the day provides a relatively comprehensive data set compared to that measured during isolated medical appointments. This approach identifies trends in a patient's blood pressure, such as a gradual increase or decrease, which may indicate a medical condition that requires treatment. The system also minimizes effects of ‘white coat syndrome’ since the monitor automatically and continuously makes measurements away from a medical office with basically no discomfort to the patient. Real-time, automatic blood pressure measurements, followed by wireless transmission of the data, are only practical with a non-invasive, cuffless system like that of the present invention. Measurements can be made completely unobtrusive to the patient.

The system can also characterize the patient's heart rate and blood oxygen saturation using the same optical system for the blood-pressure measurement. This information can be wirelessly transmitted along with blood-pressure information and used to further diagnose the patient's cardiac condition.

The monitor is easily worn by the patient during periods of exercise or day-to-day activities, and makes a non-invasive blood-pressure measurement in a matter of seconds. The resulting information has many uses for patients, medical professional, insurance companies, pharmaceutical agencies conducting clinical trials, and organizations for home-health monitoring.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF SEVERAL VIEWS OF THE DRAWINGS

FIG. 1A is a schematic view of an adhesive patch sensor system of the invention that combines electrical and optical systems to measure blood pressure and other vital signs from a patient;

FIG. 1B is a schematic view of the adhesive patch sensor system of FIG. 1A attached to the patient;

FIG. 2 is a graph of time-dependent optical and electrical waveforms generated by the adhesive patch sensor system of FIGS. 1A and 1B;

FIGS. 3A and 3B are, respectively, schematic bottom and top views of the optical system used in the adhesive patch sensor system of FIG. 1A;

FIG. 4 is an exploded view of a housing featuring top and bottom shells that house the optical system of FIG. 1A; and

FIG. 5 is a schematic view of an Internet-based system that sends vital sign information from the adhesive patch sensor system of FIG. 1A to an Internet-accessible website.

### DETAILED DESCRIPTION OF THE INVENTION

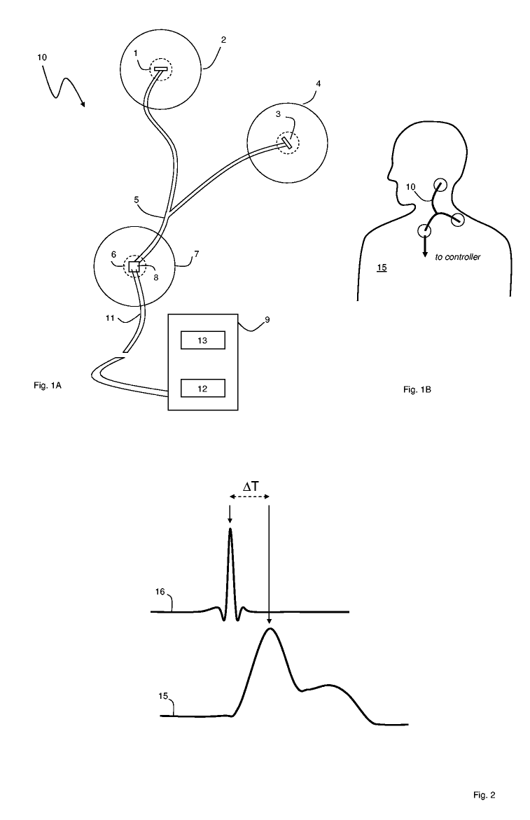

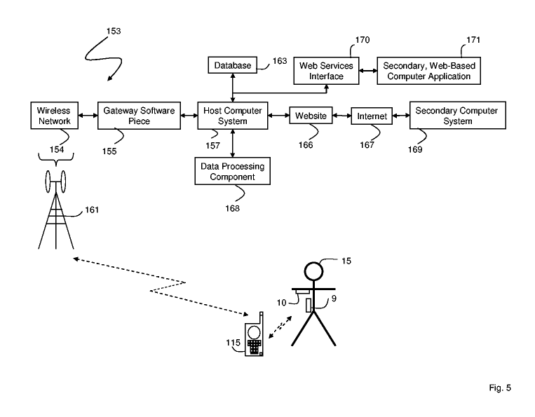

FIGS. 1A and 1B show an adhesive patch sensor system 10 according to the invention that features primary 1 and reference 3 electrodes and an optical system 6 operating in concert as described below to measure vital signs from a patient 15. The electrodes 1, 3 and optical sensor 6 each attach to the patient's skin using a separate adhesive pad 2, 4, 7, and connect to each other using a Y-shaped cable 5. During operation, the primary 1 and reference 3 electrodes detect electrical impulses, similar to those used to generate a conventional ECG, from the patient's skin. Each heartbeat generates a unique set of electrical impulses. Concurrently, the optical system 6 measures an optical waveform by detecting a time-dependent volumetric change in an underlying artery caused by blood flow following each heartbeat. The optical waveform is similar to an optical plethysmograph measured by a pulse oximeter. A circuit board 8 (described with reference to FIG. 3) attached to the optical system 6 connects on one side to the Y-shaped cable 5, and on the other side to a separate cable 11 that connects to a controller 9. The controller 9 features signal-processing electronics 12 and a microprocessor 13 that receive the electrical impulses and convert these to an electrical waveform (e.g., an ECG), and is described in more detail in U.S. patent application Ser. No. 10/906,314, filed Feb. 14, 2005 and entitled PATCH SENSOR FOR MEASURING BLOOD PRESSURE WITHOUT A CUFF, the contents of which are incorporated herein by reference. The microprocessor runs an algorithm that processes the electrical and optical waveforms as described below to measure vital signs, such as pulse oximetry, heart rate, ECG, and blood pressure.

Preferably the patch sensor system 10 attaches to a region near the patient's neck, chest, ear, or to any other location that is near the patient's head and proximal to an underling artery. Typically the patient's head undergoes relatively little motion compared to other parts of the patient's body (e.g., the hands), and thus attaching the patch sensor system 10 to these regions reduces the negative affects of motion-related artifacts. For the purposes of measuring blood pressure as described herein, the primary 1 and reference 3 electrodes only need to collect electrical signals required to generate an electrical waveform found in a 2-lead ECG. These electrodes can therefore be placed on the patient at positions that differ from those used during a standard multi-lead ECG (e.g., positions used in ‘Einthoven's Triangle’).

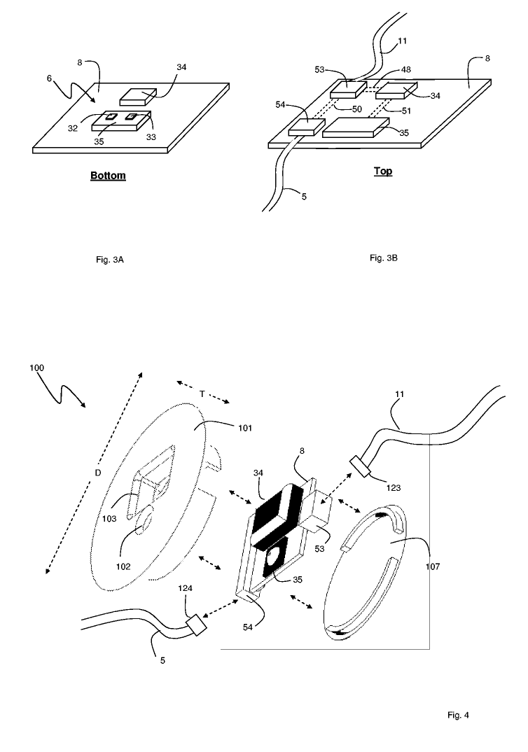

FIG. 2 shows both the optical 15 and electrical 16 waveforms generated by, respectively, the electrodes and optical system in the patch sensor system. Following a heartbeat, electrical impulses travel essentially instantaneously from the patient's heart to the electrodes, which detect it to generate the electrical waveform 16. At a later time, a pressure wave induced by the same heartbeat propagates through the patient's arteries, which are elastic and increase in volume due to the pressure wave. Ultimately the pressure wave arrives at a portion of the artery underneath the optical system, where light-emitting diodes and a photodetector detect it by measuring a time-dependent change in optical absorption to generate the optical waveform 15. The propagation time of the electrical impulse is independent of blood pressure, whereas the propagation time of the pressure wave depends strongly on pressure, as well as mechanical properties of the patient's arteries (e.g., arterial size, stiffness). The microprocessor runs an algorithm that analyzes the time difference ΔT between the arrivals of these signals, i.e. the relative occurrence of the optical 15 and electrical 16 waveforms as measured by the adhesive patch sensor.

Calibrating the measurement (e.g., with a conventional blood pressure cuff) accounts for patient-to-patient variations in arterial properties, and correlates ΔT to both systolic and diastolic blood pressure. This results in a calibration table. During an actual measurement, the calibration source is removed, and the microprocessor analyzes ΔT along with other properties of the optical and electrical waveforms and the calibration table to calculate the patient's real-time blood pressure.

The microprocessor can analyze other properties of the optical waveform 15 to augment the above-mentioned measurement of blood pressure. For example, the waveform can be ‘fit’ using a mathematical function that accurately describes the waveform's features, and an algorithm (e.g., the Marquardt-Levenberg algorithm) that iteratively varies the parameters of the function until it best matches the time-dependent features of the waveform. In this way, blood pressure-dependent properties of the waveform, such as its width, rise time, fall time, and area, can be calibrated as described above. After the calibration source is removed, the patch sensor measures these properties along with ΔT to determine the patient's blood pressure. Alternatively, the waveforms can be filtered using mathematical techniques, e.g. to remove high or low frequency components that do not correlate to blood pressure. In this case the waveforms can be filtered using well-known Fourier Transform techniques to remove unwanted frequency components.

Methods for processing the optical and electrical waveform to determine blood pressure are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL-SIGN MONITOR FOR ATHLETIC APPLICATIONS (U.S. Ser. No. ______; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); and 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); and PATCH SENSOR FOR MEASURING BLOOD PRESSURE WITHOUT A CUFF (U.S. Ser. No. 10/906,315; filed Feb. 14, 2005).

FIGS. 3A and 3B show, respectively, top and bottom views of a circuit board 8 that supports an optical system 6 featuring a light source 35 containing a pair of light-emitting diodes 32, 33 and photodetector 34. During operation, the bottom side of the optical system 6 (e.g., FIG. 3A) attaches to the patient's skin using an adhesive patch, and the light-emitting diodes 32, 33 sequentially generate red and infrared radiation that reflects off an underlying artery. The photodetector 34 detects the reflected radiation, which is digitized by an analog-to-digital converter in the controller or coupled directly with the photodetector 34 to generate an optical waveform. Concurrently, the two electrodes (shown in FIGS. 1A and 1B) generate electrical impulses that pass through the Y-shaped cable 5 to a first connector 54 mounted on the circuit board 8. The first connector 54 receives the electrical impulses and sends them through a first series of embedded traces 50 to a second connector 53. The second connector 53 also receives a signal representative of the optical waveform that passes through a second set of imbedded traces 48 from the photodetector 34. A cable 11 connects to the second connector 53 and passes the electrical impulses and signal representative of the optical waveform to the controller, which then processes this information, as described above, to measure a patient's systolic and diastolic blood pressure, heart rate, ECG, and pulse oximetry. The cable 11 also supplies power and ground to the light-emitting diodes 32, 33 and photodetector 34 through the first 48 and a third 51 series of embedded traces.

Referring to FIG. 4, a detachable housing 100 featuring bottom 101 and top 107 shells houses the circuit board 8 that supports the light source 35, photodetector 34, and first 54 and second 53 connectors. The housing 100 increases signal quality by blocking ambient light from the photodetector, and also can be easily attached to the patient's skin with an adhesive. The bottom shell 101 includes openings 102, 103 for, respectively, the light source 35 and photodetector 34. The top 107 and bottom 101 shells snap together to provide openings that provide clearance for lock-in connectors 124, 123 attached to cables 11, 5 that connect to, respectively, the first 54 and second 53 connectors.

The housing 100 preferably features a diameter ‘D’ ranging from 0.5 centimeter (‘cm’) to 10 cm, more preferably from 1.5 cm to 3.0 cm, and most preferably 2.5 cm. The housing 100 preferably has a thickness ‘T’ ranging from 2 millimeters (“mm”) to 5 mm, more preferably from 2.5 mm to 3.5 mm, and most preferably 3.0 mm. It is preferably composed of a soft, polymeric material such as a neoprene rubber, is preferably colored to match a patient's skin color, and is preferably opaque to reduce the affects of ambient light. The housing is preferably circular in shape, but can also be non-circular, e.g. an oval, square, rectangular, triangular or other shape.

FIG. 5 shows a preferred embodiment of an Internet-based system 153 that operates in concert with the adhesive patch sensor system 10 and controller 9 to send information from a patient 15 to a hand-held wireless device 115 (e.g., a conventional cell phone). The wireless device 115 then sends the information through a wireless network 154 to a web site 166 hosted on an Internet-based host computer system 157. A secondary computer system 169 accesses the website 166 through the Internet 167. The system 153 functions in a bi-directional manner, i.e. the controller 9 can both send and receive data. Most data flows from the controller 9 to the website 166; using the same network, however, the device can also receive data (e.g., ‘requests’ to measure data or text messages) and software upgrades.

A wireless gateway 155 connects to the wireless network 154 and receives data from one or more wireless devices 115, as discussed below. The wireless gateway 155 additionally connects to a host computer system 157 that includes a database 163 and a data-processing component 168 for, respectively, storing and analyzing the data. The host computer system 157, for example, may include multiple computers, software pieces, and other signal-processing and switching equipment, such as routers and digital signal processors. The wireless gateway 155 preferably connects to the wireless network 154 using a TCP/IP-based connection, or with a dedicated, digital leased line (e.g., a frame-relay circuit or a digital line running an X.25 or other protocols). The host computer system 157 also hosts the web site 166 using conventional computer hardware (e.g. computer servers for both a database and the web site) and software (e.g., web server and database software).

During typical operation, the patient continuously wears the adhesive patch sensor system 10 for a period of time ranging from a 1-2 days to weeks. Alternatively, the patient may wear the sensor 10 for shorter periods of time, e.g. just a few hours. For example, the patient may wear the sensor during a brief hospital stay, or during a medical checkup. To view information sent from the controller 9, the patient or medical professional accesses a user interface hosted on the web site 166 through the Internet 167 from the secondary computer system 169. The system 153 may also include a call center, typically staffed with medical professionals such as doctors, nurses, or nurse practioners, whom access a care-provider interface hosted on the same website 166.

In an alternate embodiment, the host computer system 157 includes a web services interface 170 that sends information using an XML-based web services link to a secondary, web-based computer application 171. This application 171, for example, could be a data-management system operating at a hospital.

The controller 9 can optionally be used to determine the patient's location using embedded position-location technology (e.g., GPS, network-assisted GPS, or Bluetooth™, 802.11-based location system). In situations requiring immediate medical assistance, the patient's location, along with relevant medical data collected by the blood pressure monitoring system, can be relayed to emergency response personnel.

In a related embodiment, the controller 9 and wireless device 115 may use a ‘store and forward’ protocol wherein one of these devices stores information when the wireless device is out of wireless coverage, and then sends this information to the wireless device when it roams back into wireless coverage.

In an alternate embodiment of the invention, the controller and adhesive patch sensor system are used within a hospital, and the controller includes a short-range wireless link (e.g., a module operating Bluetooth™, 802.11a, 802.11b, 802.1g, or 802.15.4 wireless protocols) that sends vital-sign information to an in-hospital network. In this embodiment, a nurse working at a central nursing station can quickly view the vital signs of the patient using a simple computer interface.

In still other embodiments, electronics associated with the controller (e.g., the microprocessor) are disposed directly on the adhesive patch sensor system, e.g. on the circuit board that supports the optical system. In this configuration, the circuit board may also include a display to render the patient's vital signs. In another embodiment, a short-range radio (e.g., a Bluetooth™, 802.15.4, or part-15 radio) is mounted on the circuit board and wirelessly sends information (e.g., optical and electrical waveforms; calculated vital signs such as blood pressure, heart rate, pulse oximetry, ECG, and associated waveforms) to an external controller with a matched radio, or to a conventional cellular telephone or wireless personal digital assistant. Or the short-range radio may send information to a central computer system (e.g., a computer at a nursing station), or though an internal wireless network (e.g. an 802.11-based in-hospital network). In yet another embodiment, the circuit board can support a computer memory that stores multiple readings, each corresponding to a unique time/date stamp. In this case, the readings can be accessed using a wireless or wired system described above.

In still other embodiments, the adhesive patch sensor system can include sensors in addition to those described above, e.g. sensors that measure temperature, motion (e.g. an accelerometer), or other properties. Or the sensor system can interface with other sensors, such as a conventional weight scale.

Still other embodiments are within the scope of the following claims.

1. A system for measuring vital signs from a patient, comprising:
a first adhesive patch comprising a first electrode that measures a first electrical signal from the patient;
a second adhesive patch comprising a second electrode that measures a second electrical signal from the patient;
a third adhesive patch, in electrical communication with the first and second adhesive patches, comprising an optical system that measures an optical waveform from the patient; and
a controller that receives and processes the first and second electrical signals and the optical waveform to determine the patient's vital signs.

2. The system of claim 1, wherein the optical system comprises a light-emitting diode and an optical detector.

3. The system of claim 2, wherein the optical system further comprises a substrate, and the light-emitting diode and optical detector are disposed on a same side of the substrate.

4. The system of claim 3, wherein the optical detector is aligned to detect radiation first emitted from the light-emitting diode and then reflected from the patient's skin to generate the optical waveform.

5. The system of claim 1, wherein the controller further comprises an algorithm configured to process the first and second electrical signals to generate an electrical waveform.

6. The system of claim 5, wherein the controller further comprises an algorithm that processes the electrical waveform and the optical waveform to calculate a blood pressure value.

7. The system of claim 6, wherein the controller further comprises an algorithm that determines blood pressure by processing: 1) a first time-dependent feature of the optical waveform; 2) a second time-dependent feature of the electrical waveform; and 3) a calibration parameter.

8. The system of claim 1, wherein the third adhesive patch further comprises a connector configured to connect to a detachable cable.

9. The system of claim 8, further comprising a detachable cable that connects the first electrode comprised by the first adhesive patch and the second electrode comprised by the second adhesive patch to the connector comprised by the third adhesive patch.

10. The system of claim 1, further comprising a cable that connects the third adhesive patch to the controller.

11. The system of claim 1, wherein the third adhesive patch further comprises a first wireless component, and the controller further comprises a second wireless component configured to communicate with first wireless component.

12. The system of claim 1, wherein the controller is connected directly to the third adhesive patch.

13. The system of claim 1, wherein the optical system further comprises a first light-emitting diode that emits radiation that generates a first optical waveform, and a second light-emitting diode that emits radiation that generates a second optical waveform.

14. The system of claim 13, wherein the first light-emitting diode is configured to emit red radiation, and the second light-emitting diode is configured to emit infrared radiation.

15. The system of claim 14, wherein the controller further comprises an algorithm that processes the first and second optical waveforms to generate a pulse oximetry value.

16. The system of claim 14, wherein the controller further comprises an algorithm that processes at least one of the first and second optical waveforms to generate a heart rate value.

17. The system of claim 1, wherein the controller further comprises an algorithm that processes the first and second electrical signals to generate an ECG waveform.

18. The system of claim 1, wherein the third adhesive patch further comprises a third electrode that measures a third electrical signal from the patient.

19. The system of claim 18, wherein the controller further comprises an algorithm that processes the first, second, and third electrical signals to generate an ECG waveform.
